(19) United States
(12) Patent Application Publication
(10) Pub. No.: US 2023/0210920 A1
(43) Pub. Date: Jul. 6, 2023

(54) METHODS AND COMPOSITIONS USING SERPIN PRODUCING BACTERIA

(71) Applicant: SOCIETE DES PRODUITS NESTLE S.A., Vevey (CH)

(72) Inventors: TIAGO ALVES NUNES, Bern (CH); GABRIELA BERGONZELLI DEGONDA, Bussigny (CH); STEPHANE DUBOUX, St-Prex (CH); PETER DUNCAN, Epalinges (CH); CARMINE D'URZO, Lausanne 26 (CH); ELENA VERDU DE BERCIK, Ancaster (CA)

(21) Appl. No.: 17/998,173

(22) PCT Filed: May 11, 2021
(86) PCT No.: PCT/EP2021/062407

§ 371 Date: Nov. 8, 2022

(30) Foreign Application Priority Data
May 14, 2020 (EP) 20174732.6

(51) Int. Cl. A61K 35/745; A61P 1/00; A61K 38/55; A61P 37/00

(52) U.S. Cl. CPC A61K 35/745; A61P 1/00; A61K 38/55; A61P 37/00; A61K 2035/115

(57) ABSTRACT

Serpin protein producing Bifidobacterium longum subsp. longum and compositions thereof for use in inhibiting gluten digestion. Compositions comprising the serpin protein producing Bifidobacterium longum subsp. longum may be used to mitigate the effects of hidden gluten in individuals following a gluten free diet.

### FIELD OF THE INVENTION

The present invention relates to serpin producing bacteria, and their use.

### BACKGROUND TO THE INVENTION

Gluten-related disorders comprise all diseases triggered by gluten (which includes gliadin and other proteins/peptides present in gluten), wheat and other related cereals. They include, amongst other pathophysiologies, celiac disease (CD) and non-celiac gluten/wheat sensitivity (NCGS). Currently, the incidence of a wide spectrum of gluten-related disorders is growing all around the world, especially for CD and NCGS. Both disorders are triggered by ingestion of gluten (or wheat). Both innate and adaptive immunity are implicated in CD while only innate immunity is implicated in NCGC. Alterations of intestinal barrier function were also observed in both entities.

Gluten and other related proteins from wheat and other cereals are digested in the intestine into different peptides. The best characterized peptides are those originated from gluten digestion. Certain amino acid sequences within gluten, when found in fragments of gluten following digestion (ie peptides from gluten), are recognized as toxic and/or immunogenic by the host and are to be avoided by people sensitive to the ingestion of gluten (Caminero et al., 2015, British Journal of Nutrition, 114, 1157-1167).

A life-long gluten-free diet (GFD) is the gold standard treatment for CD and NCGS patients. Following a strict GFD is very difficult due to accidental gluten intake. Low level cross-contaminations are difficult to avoid and may happen through the whole food production chain, from grain growth to manufacturing processing (Mitchison et al., 1991, Gut, 32(3), 260-265). Most of the dietary intake of gluten by people following a GFD comes from what is known as hidden gluten, i.e. little amounts of gluten present in daily meals by contamination or minor unintentional dietary mistakes. It has been described that up to 3 g of hidden gluten might be consumed daily under a strict gluten-free diet (Aziz et al., 2014, The American journal of gastroenterology, 109(9), 1498). Therefore, a solution to mitigate the effects of hidden gluten in people following a strict GFD is urgently needed

Celiac Disease (CD) is prevalent especially in the United States and Europe where around 1% of subjects had positive antibody tests (Dubé et al., 2005, Gastroenterology, 128(4), S57-S67). It is a complex disorder which arises from a complicated interaction among various immunologic, genetic, and environmental factors (Alaedini & Green, 2005). It is triggered by the digestion of wheat gluten and other related cereal proteins such as rye and barley proteins. Symptoms linked with CD are growth retardation, irritability and pubertal delay in children and many gastrointestinal symptoms such as discomfort, diarrhoea, occult stool, steatorrhea and flatulence, (Dubé et al., 2005; Sedghizadeh et al., 2002).

Non-celiac gluten sensitivity (NCGS) is an emerging condition. It is defined as a clinical entity induced by the ingestion of gluten leading to intestinal and/or extraintestinal symptoms which could be improved by removing the gluten-containing foodstuff from the diet (Lundin & Alaedini, 2012). In addition to gliadin (the main cytotoxic antigen of gluten), other proteins/peptides present in gluten and gluten-containing cereals (wheat, rye, barley, and their derivatives) may play a role in the development of symptoms. NCGS is the most common syndrome of gluten-related disorders with prevalence rates between 0.5-13% in the general population (on average 5%) (Catassi et al., 2013, Nutrients, 5(10), 3839-3853).

Dietary supplements, comprising digestive enzymes that break down gluten, are commercially available and have been proposed as a solution for dealing with occasional gluten exposure in

NCGS individuals. However, none have been shown clinically in humans to actually reduce symptoms. The aim of such digestive-enzyme based supplements is to hydrolyse the gluten. If these enzymes do not act quickly in the upper gut to extensively hydrolyse gluten, they may increase the presence of toxigenic/immunogenic gluten peptides.

Serine protease inhibitors (serpin) are a superfamily of proteins found in eukaryotes (Gettins, 2002, Chemical reviews, 102(12), 4751-4804) and prokaryotes (Kantyka et al., Biochimie, 92(11), 1644-1656).

Recently, human serine protease inhibitors have been shown to play an important role in gluten-related disorders. Elafin is human serine protease inhibitor which shows potent inhibitory capacity against various forms of elastases and proteinases (Ying & Simon, 1993, Biochemistry, 32(7), 1866-1874). Elafin is expressed throughout the epithelium of the gastrointestinal tract and its expression and induction is decreased in the intestine of patients with inflammatory bowel disease and CD (Baranger, Zani, Labas, Dallet-Choisy, & Moreau, 2011; Motta et al., 2012). Eukaryotic serpins are known to possess anti-inflammatory properties, which are linked to their ability to inhibit pancreatic elastase. Recently, elafin has been identified as a substrate for the cross-linking activity of transglutaminase 2 (TG2) (Baranger et al., 2011, PloS one, 6(6), e20976; Motta et al., Science translational medicine, 4(158), 158ra144-158ra144). In-vitro data shows that elafin moderately inhibits transglutaminase 2 (TG2) thus inhibiting the deamidation of the digestion-resistant 33-mer gliadin peptide, which is one of the potential triggers of the adaptive immune response in CD (McCarville et al. 2015, Current opinion in pharmacology, 25, 7-12).

Delivery of elafin, produced by a recombinant Lactococcus lactis has been shown to reduce gluten-induced pathology and normalise intestine inflammation in a mouse model of gluten sensitivity (Galipeau et al., 2014, The American journal of gastroenterology, 109(5), 748-756). However, this proposed therapy is based on a genetically modified microorganism (GMO) and is therefore not compatible with a food application, as consumer acceptance of GMO is very low.

More recently, serpins have been reported in prokaryotes. In silico analysis revealed the presence of genes encoding serpin-like proteins in different Bifidobacterium species, particularly in bacteria of the species Bifidobacterium longum subsp longum. The protein encoded by B. longum subsp longum (named B. longum) NCC 2705 displayed similar in-vitro antiprotease activity to those of human serpin, even so they only shared 30% identity (Ivanov et al 2006, Journal of Biological Chemistry, 281(25), 17246-17252).

B. longum NCC 2705 was deposited with the Institute Pasteur, 28 rue Dr Roux, 75724 Paris Cedex 15, France, according to the Budapest Treaty on 29 Jan. 2001 receiving the deposit no. CNCM 1-2618. B.longum NCC 2705 (CNCM 1-2618) was deposited by Nestec S.A., Avenue Nestlé 55, 1800 Vevey, Switzerland. Since then, Nestec S.A. has merged into Société des Produits Nestlé S.A. Accordingly, Société des Produits Nestlé S.A. is the successor in title of Nestec S.A., under article 2(ix) of the Budapest Treaty.

It has recently been shown that B. longum NCC 2705 (CNCM 1-2618) wild type strain and its derived recombinant strain constitutively overexpressingserpin, but not a serpin knockout mutant, attenuates gliadin-induced immunopathology in a mouse model of gluten sensitivity (NOD/DQ8 mice), (McCarville et al., 2017, Appl. Environ. Microbiol. Vol. 83, no. 19, e01323-17).

### SUMMARY OF THE INVENTION

The present inventors have surprisingly found that B. longum NCC 2705 (CNCM 1-2618) effectively inhibits the digestion of gluten, and the production of toxigenic/immunogenic peptides, in humans.

The present inventors have demonstrated for the first time that B. longum NCC 2705 (CNCM I-2618), through its ingestion by the consumer, delivers serine protease inhibitor (Serpin) to the digestive tract, and inhibits the digestion of gluten.

Advantageously, the present inventors have demonstrated for the first time that consumption by humans of a bacteria capable of producing a serine protease inhibitor can effectively reduce intestinal proteolytic activity towards gluten. Surprisingly the presence of the inhibition of glutenasic activity could be demonstrated at the hypothesized site of action (i.e. the duodenum), whereas as bifidobacteria are anaerobic bacteria, which mainly reside in the colon.

It is a clear advantage of the invention that it reduces the proteolytic activity towards gluten in the complex human intestinal milieu, at the site of action.

Furthermore, it has surprisingly been found that B. longum NCC 2705 (CNCM 1-2618) effectively inhibits gluten digestion even at a relatively low serpin level. It may be assumed that to reduce the digestion of gluten, a relatively large amount of protease inhibitor would be required (e.g. approximately 1:1 ratio of inhibitor:protease), given that serpins are classed as irreversible inhibitors (also known as suicide inhibitors) and can only function once (Gettins P G. 2002 Chem Rev. 102(12):4751-804). It is a further advantage of the invention that B. longum NCC 2705 (CNCM 1-2618) can effectively reduce the digestion of gluten even at a relatively small amount of serpin in the site of action.

Accordingly, in a first aspect of the invention there is provided a composition comprising a therapeutically effective amount of a serpin protein producing Bifidobacterium longum subsp longum for use in inhibiting digestion of gluten in an individual in need thereof.

In a related aspect there is provided a composition comprising a therapeutically effective amount of a serpin protein producing Bifidobacterium longum subsp longum for use in inhibiting production of toxigenic/immunogenic peptides from gluten.

In an embodiment of the invention there is provided a composition comprising a therapeutically effective amount of a serpin protein producing Bifidobacterium longum subsp longum for use in the prevention and/or treatment of symptoms of accidental/hidden gluten ingestion in an individual on a GFD. In an embodiment the individual is an individual with CD.

In another aspect of the invention there is provided a composition comprising a therapeutically effective amount of a serpin protein producing Bifidobacterium longum subsp longum for use in combination with a GFD in the treatment of CD.

The composition is for enteral administration, preferably oral administration. In one embodiment, the composition is a food, a medical food, a tube feed or a dietary supplement. In a specific embodiment the composition is a dietary supplement.

In one embodiment, the food is selected from milk, yoghurt, curd, cheese, fermented milks, milk based fermented products, rice and other non-gluten containing cereal based products, milk based powders, infant formulae and pet food.

In one embodiment, the composition is a pharmaceutical composition wherein the pharmaceutical composition comprises one or more pharmaceutically acceptable carriers, diluents and/or excipients.

In an embodiment the composition is a dietary supplement, wherein the dietary supplement is in the form of a tablet, a capsule, a lozenge or a powder.

It has surprisingly been found that B. longum NCC 2705 (CNCM 1-2618) effectively inhibits gluten digestion even at a relatively low serpin level. It is a further advantage of the invention that a Bifidobacterium longum subsp longum producing a small amount of serpin can effectively reduce the digestion of gluten.

In an embodiment the composition provides from about 10⁶ cfu (Colony forming units) to 10¹² cfu of the serpin protein producing Bifidobacterium longum subsp longum strain per dose of the composition, preferably from about 10⁸ cfu to about 10¹² cfu, more preferably from about 10⁹ cfu to about 10¹¹ cfu, such as about 10¹⁰ cfu of the a serpin protein producing Bifidobacterium longum subsp longum per dose of the composition. In an embodiment the composition provides from about 10⁶ cfu to about 10¹² cfu Bifidobacterium longum subsp longum strain CNCM 1-2618 (NCC 2705) per dose of the composition, preferably from about 10⁸ cfu to about 10¹² cfu, more preferably about 10⁹ cfu to about 10¹¹ cfu, Bifidobacterium longum subsp longum strain CNCM I-2618 (NCC 2705) per dose of the composition. In an embodiment the composition provides from about 10¹⁰ cfu Bifidobacterium longum subsp longum strain CNCM 1-2618 (NCC 2705) per dose of the composition

The composition can be administered to the individual in a daily dose comprising between 10⁶ and 10¹² cfu of the serpin protein producing Bifidobacterium longum subsp longum strain, preferably from about 10⁶ cfu to about 10¹² cfu, more preferably from about 10⁹ cfu to about 10¹¹ cfu, such as about 10¹⁰ cfu of a serpin protein producing Bifidobacterium longum subsp longum.

In an embodiment the composition can be administered to the individual in a daily dose comprising between 10⁶ and 10¹² cfu Bifidobacterium longum subsp longum strain CNCM I-2618 (NCC 2705) preferably from about 10⁸ cfu to about 10¹² cfu, more preferably about 10⁹ cfu to about 10¹¹ cfu Bifidobacterium longum subsp longum strain CNCM 1-2618 (NCC 2705). In an embodiment the composition is administered to the individual in a daily dose of about 10¹⁰ cfu Bifidobacterium longum subsp longum strain CNCM 1-2618 (NCC 2705).

In an embodiment, the composition is administered to the individual each day of a time period that is at least three days, preferably at least four days.

The daily dose of the composition can be administered to the individual in a once daily administration, a twice daily administration, a three times daily administration or a four times daily administration.

In an embodiment the composition is administered to the individual twice daily, for example in the morning and in the evening.

In another aspect of the invention there is provided a serpin protein producing Bifidobacterium longum subsp longum for use in inhibiting gluten digestion in an individual with CD.

In another aspect of the invention there is provided a serpin protein producing Bifidobacterium longum subsp longum for use in inhibiting production of toxigenic/immunogenic peptides from gluten in an individual with CD.

In an embodiment the individual is on a GFD.

In an embodiment there is provided a serpin protein producing Bifidobacterium longum subsp longum for use in the prevention and/or treatment of symptoms of accidental/hidden gluten ingestion in an individual with CD.

The serpin protein producing Bifidobacterium longum subsp longum is preferably selected from Bifidobacterium longum subsp longum strain CNCM 1-2169, Bifidobacterium longum subsp longum strain CNCM 1-2171, Bifidobacterium longum subsp longum strain ATCC BAA-999, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM 1-2618 (NCC 2705), Bifidobacterium longum subsp longum strain CNCM 1-2170, Bifidobacterium longum subsp longum strain ATCC 15707 (T), or a combination thereof. In a preferred embodiment the serpin protein producing Bifidobacterium longum subsp longum is Bifidobacterium longum subsp longum strain CNCM I-2618 (NCC 2705).

In another aspect of the invention there is provided a combination of (i) a serpin protein producing Bifidobacterium longum subsp longum and (ii) GFD, for use in the treatment of CD

In an embodiment the combination or composition according to the invention may be used for the treatment of CD in individual with CD following a GFD and experiencing persistence of symptoms.

In an embodiment the composition or combination according to the invention may be used for prevention or prophylaxis of symptoms, due to consumption of a small amount of gluten (e.g. up to 5 g of gluten, preferably up to 3 g of gluten, per consumption incident), in an individual with CD.

In an embodiment the composition or combination according to the invention may be used for prevention or prophylaxis of symptoms, due to accidental/unintended consumption of gluten, in an individual with CD.

In other aspect there is provided a composition comprising a serpin protein producing Bifidobacterium longum subsp longum in an amount clinically demonstrated in a double-blinded, placebo controlled, double crossover study to inhibit gluten digestion in an individual with CD.

In an embodiment there is provided a composition comprising a serpin protein producing Bifidobacterium longum subsp longum in an amount clinically demonstrated in a double-blinded, placebo controlled study to inhibit gluten digestion in an individual CD following ingestion of an amount of gluten corresponding to hidden gluten consumption in a gluten-free diet.

In another aspect there is provided a method of inhibiting gluten digestion in an individual in need thereof, comprising administering a therapeutically effective amount of serpin protein producing Bifidobacterium longum subsp longum to the individual in need thereof.

In an embodiment there is provided a method of inhibiting production of toxigenic/immunogenic gluten peptides in an individual in need thereof comprising administering a therapeutically effective amount of a serpin protein producing Bifidobacterium longum subsp longum to an individual in need thereof.

In another aspect there is provided a method for the treatment of CD comprising administering to an individual in need thereof a therapeutically effective amount of a serpin protein producing Bifidobacterium longum subsp longum clinically demonstrated in a double-blinded, placebo controlled, crossover study, to inhibit gluten digestion.

In an embodiment the individual is a CD patient suffering from persistence of symptoms on a GFD.

In some embodiments the Bifidobacterium longum subsp longum may be selected from from Bifidobacterium longum subsp longum strain CNCM 1-2169, Bifidobacterium longum subsp longum strain CNCM 1-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM 1-2618 (NCC 2705), Bifidobacterium longum subsp longum strain CNCM 1-2170, Bifidobacterium longum subsp longum strain ATCC 15707 (T), or a combination thereof.

In some preferred embodiments, the Bifidobacterium longum subsp longum may be selected from Bifidobacterium longum subsp longum strain CNCM 1-2169, Bifidobacterium longum subsp longum strain CNCM 1-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM 1-2618 (NCC 2705), Bifidobacterium longum subsp longum strain CNCM 1-2170, Bifidobacterium longum subsp longum strain ATCC 15707 (T), or a combination thereof.

In preferred embodiments, the Bifidobacterium longum subsp longum strain CNCM 1-2618 (NCC 2705) is used.

An advantage of one or more embodiments provided by the present disclosure is a composition comprising a bacterial strain that is effective, and safe to administer without unwanted side effects, which can be used to treat or prevent the negative effects of accidental consumption of gluten in an individual with CD.

A further advantage of the present disclosure is to allow CD patients on a GFD to cope with hidden gluten.

Another advantage of one or more embodiments provided by the present disclosure is to provide a better safety profile relative to known enzymatic supplements.

A further advantage of one or more embodiments provided by the present disclosure is to minimize or avoid completely the side effects from accidental/hidden gluten consumption.

An additional advantage of one or more embodiments provided by the present disclosure is to improve the effect of a GFD in the treatment of CD.

Yet another advantage of one or more embodiments provided by the present disclosure is to minimize or avoid completely unnecessary costs related to healthcare assistance.

Another advantage of one or more embodiments provided by the present disclosure is to achieve effective inhibition of gluten digestion with a Bifidobacterium longum subsp longum probiotic producing a low level of serpin.

Additional features and advantages are described herein and will be apparent from the following Detailed Description and the Figures.

### DESCRIPTION OF THE DRAWINGS

FIG. 1—Shows a schematic representation of the clinical study design.

FIG. 2—shows B. longum NCC 2705 (A) and B. longum serpin protein (B) levels in human duodenum aspirates following administration of B. longum NCC 2705 or placebo in celiac disease (CD) and non-celiac gluten sensitive (NCGS) individuals.

FIG. 3—Shows the influence of B. longum NCC 2705 on glutenasic activity in CD or NCGS individuals. Values shown as AUC T0 to T370.

### DETAILED DESCRIPTION OF THE INVENTION

As used in this disclosure and the appended claims, the singular forms “a,” “an” and “the” include plural referents unless the context clearly dictates otherwise. Thus, for example, reference to “a bacterial strain” or “the bacterial strain” includes two or more bacterial strains.

The words “comprise,” “comprises” and “comprising” are to be interpreted inclusively rather than exclusively. Likewise, the terms “include,” “including” and “or” should all be construed to be inclusive, unless such a construction is clearly prohibited from the context.

Nevertheless, the compositions disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein.

Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

As used herein, “about” and “approximately” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number.

Furthermore, all numerical ranges herein should be understood to include all integers, whole or fractions, within the range. The term “between” includes the end points of the identified range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein, the terms “individual” and “patient” are understood to include an animal, especially a mammal, and more especially a human that is receiving or intended to receive treatment, as treatment is herein defined. The terms “individual” and “patient” are used herein to refer to a human. Accordingly, the terms “individual” and “patient” refer to any human that can benefit from the treatment.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e. arresting the development of the condition or disorder or its clinical symptoms and (2) relieving the condition or disorder, i.e. causing the temporary or permanent regression of the condition or disorder or its clinical symptoms. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

### Composition

The composition may be orally and/or enterally administrable. The composition of the present invention may be in the form of a food, a medical food, a tube feed, a nutritional composition, or a nutritional supplement. The term “dietary supplement” refers to a product which is intended to supplement the general diet of a subject.

In one embodiment, the food is selected from milk, yoghurt, curd, cheese, fermented milks, milk based fermented products, rice and other non-gluten containing cereal based products, milk based powders, infant formulae and pet food.

The composition may be in the form of a medical food. The term “medical food” as used herein refers to a food product specifically formulated for the dietary management of a medical disease or condition. The medical food may be administered under medical supervision. The medical food may be for oral ingestion or tube feeding.

The composition may be in the form of a tube feed. The term “tube feed” refers to a product which is intended for introducing nutrients directly into the gastrointestinal tract of a subject by a feeding tube. A tube feed may be administered by, for example, a feeding tube placed through the nose of a subject (such as nasogastric, nasoduodenal, and nasojejunal tubes), or a feeding tube placed directly into the abdomen of a subject (such as gastrostomy, gastrojejunostomy, or jejunostomy feeding tube).

The composition may be in the form of a pharmaceutical composition and may comprise one or more suitable pharmaceutically acceptable carriers, diluents and/or excipients.

Examples of such suitable excipients for compositions described herein may be found in the “Handbook of Pharmaceutical Excipients”, 2nd Edition, (1994), Edited by A Wade and P J Weller. Acceptable carriers or diluents for therapeutic use are well known in the pharmaceutical art, and are described, for example, in “Remington's Pharmaceutical Sciences”, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s) and/or solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

Preservatives, stabilisers, dyes and even flavouring agents may be provided in the composition. Examples of preservatives include sodium benzoate, sorbic acid and esters of p-hydroxybenzoic acid. Antioxidants and suspending agents may be also used.

Nutritionally acceptable carriers, diluents and excipients include those suitable for human or animal consumption that are used as standard in the food industry. Typical nutritionally acceptable carriers, diluents and excipients will be familiar to the skilled person in the art.

The composition may be in the form of a tablet, dragee, lozenges, capsule, gel cap, powder, granule, solution, emulsion, suspension, coated particle, spray-dried particle or pill.

In a preferred embodiment the composition is a dietary supplement.

In an alternative embodiment the composition may be in the form of a composition for topical administration, such as a gel, cream, ointment, emulsion, suspension or solution for topical administration.

It is clear to those skilled in the art that an ideal dose will depend on the subject to be treated, its health condition, sex, age, or weight, for example, and the route of administration. The dose to be ideally used will consequently vary but can be determined easily by those of skill in the art.

However, generally, it is preferred if the composition of the present invention comprises between 10⁶ and 10¹⁰ cfu and/or between 10⁶ and 10¹⁰ cells of Bifidobacterium longum subsp longum per daily dose. It may also comprise between 10⁶ and 10¹¹ cfu and/or between 10⁶ and 10¹¹ cells of Bifidobacterium longum subsp longum per g of the dry weight of the composition.

### Bifidobacterium Longum

The Bifidobacterium longum may be any Bifidobacterium longum subsp longum strain. In some embodiments the Bifidobacterium longum subsp longum strain may be selected from Bifidobacterium longum subsp longum strain CNCM 1-2169, Bifidobacterium longum subsp longum strain CNCM 1-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM 1-2618 (NCC 2705), Bifidobacterium longum subsp longum strain CNCM 1-2170, Bifidobacterium longum subsp longum strain ATCC 15707 (T), Bifidobacterium longum subsp longum strain CNCM 1-103, Bifidobacterium longum subsp longum strain CNCM 1-2334, Bifidobacterium longum subsp longum strain CNCM 1-3864, Bifidobacterium longum subsp longum strain CNCM 1-3853, or a combination thereof.

The strains have been deposited in the depositary institution indicated in the table below (Table 1), and have received the following date of deposit and accession number:

TABLE 1

| | Depositary | Accession | Date of |
|---|---|---|---|
| # | institution | number | deposit |
| 1 | CNCM | I-2169 | Mar. 15, 1999 |
| 2 | CNCM | I-2171 | Mar. 15, 1999 |
| 3 | ATCC | 15708 | <1990 |
| 4 | DSM | 20097 | <1990 |
| 5 | NCIMB | 8809 | Oct. 01, 1956 |
| 6 | CNCM | I-2618 | Jan. 19, 2001 |
| 7 | CNCM | I-2170 | Mar. 15, 1999 |
| 8 | ATCC | 15707 | <1990 |
| 9 | CNCM | I-103 | Oct. 29, 1979 |
| 11 | CNCM | I-2334 | Oct 12, 1999 |
| 12 | CNCM | I-3864 | Nov. 15, 2007 |
| 13 | CNCM | I-3853 | Oct. 16, 2007 |

CNCM refers to Collection nationale de cultures de micro-organismes, Institut Pasteur, 28, rue du Dr Roux, F-75724 Paris Cedex 15, France. ATCC refers to American Type Culture Collection 10801 University Blvd., Manassas, Va. 20110-2209, U.S.A. DSM refers to Leibniz Institute DSMZ-German Collection of Microorganisms and Cell Cultures, Inhoffenstr. 7B, D-38124 Braunschweig, Germany. NCIMB refers to NCIMB Ltd, Ferguson Building, Craibstone Estate, Buckburn, Aberdeen AB21 9YA, Scotland.

Strains 1, 2, 6, 7, 9, 11-13 have been deposited by Nestec S.A., avenue Nestlé 55, 1800 Vevey, Switzerland. Since then, Nestec S.A. has merged into Société des Produits Nestlé S.A. Accordingly, Société des Produits Nestlé S.A. is the successor in title of Nestec S.A., under article 2(ix) of the Budapest Treaty. All other strains are commercially available.

In some preferred embodiments, the Bifidobacterium longum subsp longum may be selected from Bifidobacterium longum subsp longum strain CNCM 1-2169, Bifidobacterium longum subsp longum strain CNCM 1-2171, Bifidobacterium longum subsp longum strain ATCC 15708, Bifidobacterium longum subsp longum strain DSM 20097, Bifidobacterium longum subsp longum strain NCIMB 8809, Bifidobacterium longum subsp longum strain CNCM 1-2618 (NCC 2705), Bifidobacterium longum subsp longum strain CNCM I-2170, Bifidobacterium longum subsp longum strain ATCC 15707 (T), or a combination thereof.

In preferred embodiments, the Bifidobacterium longum subsp longum strain B. longum CNCM I-2618 (NCC 2705) is used.

In an embodiment, at least a part of the serpin protein producing Bifidobacterium longum subsp longum according to the invention are alive in the composition and preferably arrive alive in the intestine. For example, at least 5%, preferably at least 10%, more preferably at least 15% of the serpin protein producing Bifidobacterium longum subsp longum can be viable in the composition. As a result, the alive Bifidobacterium longum subsp longum can persist in the intestine and may increase their effectiveness by multiplication. The alive Bifidobacterium longum subsp longum may also be effective by interacting with the commensal bacteria and/or the host.

In therapeutic applications, the composition is administered in an amount sufficient to at least partially cure or arrest the symptoms of the condition and its complications. An amount adequate to accomplish this purpose is defined as “a therapeutically effective dose”. Amounts effective for this purpose will depend on a number of factors known to those of skill in the art, such as the severity of the condition and the weight and general state of the patient.

In prophylactic applications, the composition can be administered to a patient susceptible to or otherwise at risk of a particular condition in an amount that is sufficient to at least partially reduce the risk of developing the condition. Such an amount is “a prophylactically effective dose.” Again, the precise amounts depend on a number of patient-specific factors, such as the patient's state of health and weight.

The composition is preferably administered in an amount that provides a therapeutically effective dose and/or in a prophylactic effective dose of the serpin protein producing Bifidobacterium longum subsp longum.

A daily dose of the composition preferably provides between 10⁶ and 10¹² cfu (colony forming units) of the serpin protein producing Bifidobacterium longum subsp longum, more preferably from 10⁸ to 10¹² cfu, most preferably from 10⁹ to 10¹¹ cfu. The composition may comprise between 10⁶ and 10¹² cfu, preferably 10⁸ to 10¹² cfu, more preferably 10⁹ to 10¹¹ cfu of the Bifidobacterium longum subsp longum per dose of the composition.

The composition may be a powder having a water activity less than 0.2, preferably less than 0.15. The composition may be a shelf-stable powder. The low water activity can provide this shelf stability and can ensure that the Bifidobacterium longum subsp longum will remain viable even after long storage times. Water activity (aw) is a measurement of the energy status of the water in a system and is defined as the vapor pressure of water divided by that of pure water at the same temperature; therefore, pure distilled water has a water activity of exactly one.

Additionally or alternatively, the serpin protein producing Bifidobacterium longum subsp longum may be provided in an encapsulated form. Encapsulation of the bacteria can have therapeutical and technical advantages. For example, encapsulation can increase the survival of the bacteria and thus the number of live bacteria which arrive in the intestine. Furthermore, the bacteria can be gradually released, allowing a prolonged action of the bacteria on the health of the subject. For example, the bacteria may be freeze or spray dried and incorporated into a gel.

### Process for Producing a Culture Powder

Strains belonging to the species B. longum are grown in anaerobic conditions. Fermentation methods under anaerobic conditions are commonly known. The skilled person is able to identify suitable components of the fermentation medium and to adjust fermentation conditions based on his general knowledge, depending on the microorganism to be grown. The fermentation medium typically comprises
a nitrogen source such as yeast extract,
a carbon source such as a sugar,
various growth factors (e.g minerals, vitamins etc.) required by the microorganism and
water.

A non-limiting example of a typical growth medium for B. longum is MRS (De Man, Rogosa and Sharpe) medium, supplemented with 0.05% of cysteine (MRSc).

The fermentation is preferably carried out in two steps, a starter fermentation being carried out prior to the main fermentation step. The fermentation medium can be different for the starter and the main fermentation or may be identical.

The second step of the process is the concentration of the biomass. This can also be carried out using methods known to the person skilled in the art, such as for example centrifugation or filtration. The total solid content of the biomass after concentration is preferably comprised between 10 and 35wt %, preferably between 14 and 35wt %, based on the total dry weight of the biomass (i.e. of the total amount of fermentation medium and produced microorganism).

Optionally, the concentration may be preceded or combined with a washing step to remove residues of the fermentation medium and/or compounds produced during fermentation. For example, washing may be performed by concentrating biomass, re-suspending the concentrated biomass in a buffer, such as a phosphate buffer, or a similar composition and re-concentrating the biomass.

For example, the process described in WO2017/001590, which is entirely incorporated by reference, can be applied.

### Treatment

### Celiac Disease (CD)

CD is one of the most common immune mediated disorders. It is a worldwide condition and is prevalent especially in the United States and Europe where around 1% of subjects had positive antibody tests. CD is a complex disorder which arises from a complicated interaction among various immunologic, genetic, and environmental factors. It is triggered by the digestion of wheat gluten and other related cereal proteins such as rye and barley proteins. Symptoms linked with CD are growth retardation, irritability and pubertal delay in children and many gastrointestinal symptoms like discomfort, diarrhoea, occult stool, steatorrhea flatulence.

Clinical evidence shows class II human leukocyte antigens (HLA-DQII), which strongly relate with CD pathology, are expressed in about 95% of CD patients. In the intestinal lumen, gluten protein are partially digested, forming proteolytic-resistant immunogenic 33-mer gluten peptide. After crossing the small intestinal barrier, they are deamidated by transglutaminase 2 (TG2) with negative charges (Sollid, 2000, Annual review of immunology, 18(1), 53-81), which then bind to the positively charged binding sites of HLA-DQ2.5/8 (Dieterich et al., 1997, Nature medicine, 3(7), 797-801). HLA-DQ2.5/8 displaying those specific gluten peptides signals to helper T cells and other immune cells causing further damage in the small intestine. Antibodies against gluten proteins and autoantibodies to connective tissue components (TG2) are also associated with CD progression (Alaedini & Green, 2005, Annals of internal medicine, 142(4), 289-298).

### Gluten Free Diet (GFD)

GFD refers to the complete withdrawal of gluten from the diet. GFD is the standard treatment for gluten sensitive individuals. Following a strict GFD is not an easy task. Patients undergoing GFD can still be exposed to varied quantities of gluten due to cross-contamination or dietary mistakes. There is a remarkable degree of variability among gluten sensitive persons with respect to their individual sensitivity to gluten, with some patients having histological changes due to a very low daily gluten exposure. Most of the dietary intake of gluten by people following a GFD comes from what is known as hidden gluten, i.e. little amounts of gluten present in daily meals by contamination or minor unintentional dietary mistakes. The present invention provides a solution to mitigate the effects of hidden gluten in people following a strict GFD.

The Bifidobacterium longum subsp longum strains according to the present invention, or a composition comprising the same, may be used to mitigate the effects of hidden glutted in individuals following a strict GFD, such as individuals with CD.

The Bifidobacterium longum subsp longum strains according to the present invention, or a composition comprising the same, may be used to inhibit digestion of gluten in an individual in need thereof, and thus inhibit production of toxigenic/immunogenic peptides from gluten.

The invention the Bifidobacterium longum subsp longum strains according to the present invention, or a composition comprising the same, may be used in the prevention and/or treatment of symptoms of accidental/hidden gluten ingestion in an individual with CD.

For example, a composition comprising a therapeutically effective amount of a serpin protein producing Bifidobacterium longum subsp longum according to the invention may be used in combination with a GFD in the treatment of CD.

The combination of (i) a serpin protein producing Bifidobacterium longum subsp longum strains according to the invention and (ii) GFD may be used in the treatment CD, for example in the treatment of CD in individual with CD following a GFD and experiencing persistence of symptoms.

The Bifidobacterium longum subsp longum strains according to the present invention, or a composition comprising the same, may be used for prevention or prophylaxis of symptoms, due to accidental/unintended consumption of gluten, in an individual with CD.

### Administration

The Bifidobacterium longum subsp longum or composition described herein are preferably administered enterally.

Enteral administration may be oral, gastric, and/or rectal.

In general terms, administration of the combination or composition described herein may, for example, be by an oral route or another route into the gastro-intestinal tract, for example the administration may be by tube feeding. Preferably administration is oral.

In an alternative embodiment administration of the combination or composition described herein may be topical administration.

The subject may be a mammal such as a human, canine, feline, equine, caprine, bovine, ovine, porcine, cervine and primates. Preferably the subject is a human.

Preferred features and embodiments of the invention will now be described by way of non-limiting examples.

### EXAMPLE

### In Vivo Biological Activity of Serpin Producing Bifidobacterium longum NCC 2705 (CNCM I-2618) in Gluten Sensitive Individuals

### Study Design:

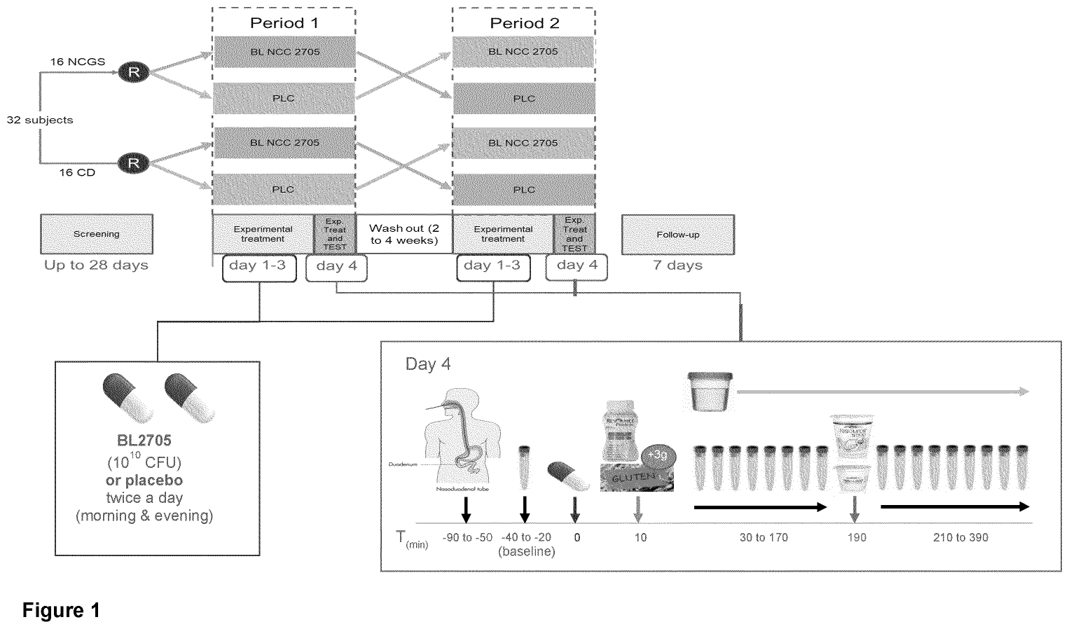

Biological activity of serpin producing Bifidobacterium longum NCC 2705 (CNCM 1-2618) was assessed in a double-blinded, randomized, placebo controlled, cross-over trial in 18 CD subjects who kept a GFD for ≥1 y and 20 self-reported NCGS subjects who kept a GFD for ≥6 wks. Subjects were aged between 18 and 65 years old, and had BMI within the range of 18-30 kg/m2. One capsule of B. longum NCC2705 (one capsule containing 1.0×10¹⁰ colony forming unit (cfu) of the probiotic strain B. longum NCC2705, premixed with a carrier which was maltodextrin) or placebo (maltodextrin) was given twice a day (morning and evening) with a meal, over 2 periods of 3 days each. On day 4 of both cross-over periods, a naso-duodenal aspiration catheter reaching the distal duodenum was placed by gastroduodenoscopy. After catheter positioning, participants received a final dose of placebo or B. longum NCC2705 (T0), followed by a 3 g-gluten challenge. Duodenal aspirates were collected at 20-minute intervals up to 370 minutes. B. longum NCC2705 level in the aspirates (genome copies/mL aspirate) was determined by qPCR and serpin protein levels in the aspirates (pg/mL aspirate) were determined by ELISA. Glutenasic activity in the aspirates was determined using gluten as substrate. A schematic representation of the study design is shown in FIG. 1.

### Results:

Gastrointestinal tolerability of the probiotics and of placebo did not differ between the groups.

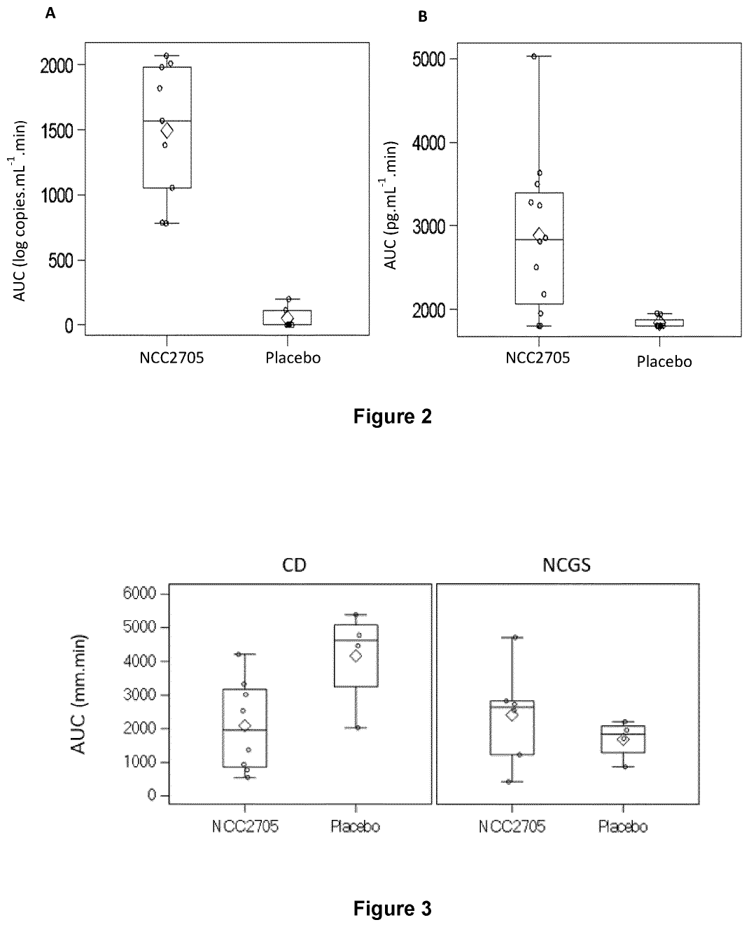

The appearance of B. longum NCC2705 in duodenal aspirates is associated with a concomitant increase in serpin concentration. The cumulated serpin over time was higher in the probiotic (Active) group than in placebo group in CD (AUC_{T0-T370} 2884.7±936.0 vs 1842.7±65.4 (pg/mL)*min; p=0.063) (FIG. 2). In NCGS, no significant difference was observed. Appearance of B. longum NCC2705 genome copies (gc) and serpin (pg) in duodenal aspirates was significantly higher in the probiotics group (pooled CD and NCGS groups), compared to placebo (BL NCC2705 AUC_{T0-T370} 1426.8±507.2 vs 132.1±182.9 (gc/mL)*min, p=0.016; serpin AUC_{T0-T370} 2654.6±821.2 vs 1860.7±137.3 (pg/mL)*min, p=0.016).

The concentration peak (Cmax) of B. longum NCC2705 gc appeared 90 min in average after product intake in both CD (6.3±1.9 gc/mL) and NCGS (6.9±1.2 gc/mL). Similarly, Serpin Cₘₐₓ was reached 90 min in average after product intake in CD (22.5±49.3 vs 4.9±0.0 pg/mL; p<0.05). In NCGS, the serpin Cmax was also significantly higher compared to placebo (25.6±24.8 vs 7.4±3.9 pg/mL; p<0.05).

As seen in FIG. 2, following and paralleling B. longum CNCM 1-2618 (B. longum NCC2705) consumption, surprisingly serpin was found in the duodenum of celiac disease patients. The presence of B. longum CNCM 1-2618 (B. longum NCC2705) and/or serpin in the duodenum may confer advantageous properties such as reduced digestion of gluten by reduction of gluten-directed proteolytic activity.

To determine glutenasic activity in the duodenal aspirates, samples were incubated on 1% gluten (Sigma-Aldrich)-containing agar plates. Proteolytic activity was determined by the presence of a halo surrounding the inoculation site.

Glutenasic acitivity in duodenal aspirates was lower in the probiotic (Active) group vs placebo group in CD subjects (AUC_{T0-T370} 2091.6±1362.5 vs 4165.7±1475.4 mm*min;). In NCGS subjets, no statistically significant difference was observed. See FIG. 3. These results demonstrate efficacy of serpin producing probiotic B. longum NCC2705 in decreasing gluten digestion in CD subjects. The consumption of B. longum CNCM 1-2618 (B. longum NCC2705) and the reduction of glutenasic activity may confer advantageous properties such as reduced digestion of gluten by reduction of gluten-directed proteolytic activity.

These results provide basis for the potential use of B. longum NCC2705 as a complementary treatment to GFD in patients still experiencing symptoms due to accidental gluten intake.

1-2. (canceled)

3. A method for use in inhibiting digestion of gluten in an individual in need thereof comprising administering a composition comprising a therapeutically effective amount of a serpin protein producing Bifidobacterium longum subsp longum to the individual.

4. A method according to claim 3 for use in inhibiting production of toxigenic and/or immunogenic peptides from gluten.

5. A method according to claim 3 wherein the individual is an individual with celiac disease.

6-8. (canceled)

9. A method according to claim 3, wherein the composition is in a form selected from the group consisting of a food, a medical food, a dietary supplement and a pharmaceutical composition

10. A method according to claim 3, wherein the composition is in a form selected from the group consisting of a tablet, a capsule, a lozenge and a powder.

11. A method according to claim 3, wherein the composition provides from 10⁶ cfu to 10¹² cfu of the Bifidobacterium longum subsp longum strain CNCM I-2618 (NCC 2705) per dose of the composition.

12. A method according to claim 11, wherein the composition is for administration twice daily.

13-16. (canceled)

17. A method for inhibiting gluten digestion, and/or inhibiting production of toxigenic and/or immunogenic gluten peptides, in an individual in need thereof, comprising administering a therapeutically effective amount of serpin protein producing Bifidobacterium longum subsp longum to the individual in need thereof.

18. A method for the treatment of celiac disease comprising administering to an individual in need thereof a therapeutically effective amount of a serpin protein producing Bifidobacterium longum subsp longum clinically demonstrated in a double-blinded, placebo controlled study, to inhibit gluten digestion.

19. A method according to claim 17, wherein the individual is a celiac disease patient.

20-21. (canceled)
